(19) United States
(12) Patent Application Publication
(10) Pub. No.: US 2021/0379552 A1
(43) Pub. Date: Dec. 9, 2021

(54) SYSTEMS METHODS AND COMPUTATIONAL DEVICES FOR AUTOMATED CONTROL OF INDUSTRIAL PRODUCTION PROCESSES

(71) Applicant: VAYU SENSE AG, Munich (DE)

(72) Inventors: Moria SHIMONI, Petach Tikva (IL); Eran AZMON, Rosh Ha'Ayin (IL); Yaakov RAZ KFIREL, Pardes Hanna-Karkur (IL); Ben HAROSH, Lod (IL)

(73) Assignee: VAYU SENSE AG, Munich (DE)

(21) Appl. No.: 17/057,266

(22) PCT Filed: Nov. 4, 2019
(86) PCT No.: PCT/IL2019/051206

§ 371 Date: Nov. 20, 2020

(30) Foreign Application Priority Data
Nov. 4, 2018 (IL) 262742

(51) Int. Cl. B01J 19/00; C12M 1/36; G05B 13/04; G05B 13/02

(52) U.S. Cl. CPC B01J 19/0033; C12M 41/48; B01J 2219/00243; G05B 13/0265; B01J 2219/00195; G05B 13/042

(57) ABSTRACT

A system and method for optimized industrial production using machine learning. The method includes creating a model defining dependencies among a plurality of parameters for an industrial production process, the plurality of parameters including a plurality of controlled parameters and a plurality of monitored parameters; training an agent via reinforcement learning based on iterative application of the model, wherein the agent is trained to determine new values for the plurality of controlled parameters based on current values of the plurality of monitored parameters in order to optimize the industrial production process with respect to at least one predetermined objective; and iteratively modifying, by the trained agent, current values of the plurality of controlled parameters in real-time during operation of the industrial production process.

### CROSS-REFERENCE TO RELATED APPLICATIONS

This application claims priority under 35 U.S.C. § 371 to the International Application No. PCT/IL2019/051206 filed on Nov. 4, 2019, now pending. The PCT/IL2019/051206 Application claims the benefit of Israeli Patent Application No. 262742 filed on Nov. 4, 2018. All of the applications referenced above are hereby incorporated by reference.

### TECHNICAL FIELD

The present disclosure relates generally to controllers for industrial production processes, and more specifically to utilizing machine learning to accurately control industrial production processes.

### BACKGROUND

Industrial production processes, such as fermentation or chemical reactor processes, are technically challenging to control. The process variables are often difficult to measure, the “quality” of the product that may be difficult to define yet very important, the process model usually contains strongly time-varying parameters, etc. Optimization of industrial fermentation processes, such as batch and fed-batch, typically depends upon optimizing the culture dynamics. Optimizing a fermenter or bioreactor-based process, such as that of a recombinant product, may be achieved by knowing and optimizing the culture state, determining the best time for induction by real-time and sensitive measurement, identifying harvesting time, etc. However, in fed-batch processes, the challenge arises because the optimization of the feed rate is a dynamical problem.

The main mission of the biotech team running developed manufacturing systems is to continuously increase yields and decrease costs. In fermentation processes of microorganisms releasing metabolites, the yield, quality and reproducibility of the production culture rely heavily on the monitoring and control of the culture kinetics in growth phase and production phase. The more the culture is under control, the better yield and quality are achievable. There is a continuous need in fermentation-based processes to increase profitability by increasing the yield and reduce fermentation time (up-stream production time).

Fermentation technology is widely used for the production of various economically important compounds which have applications in the energy production, pharmaceutical, chemical and food industry. Although fermentation processes have been used for generations, the need for sustainable production of products that meet market requirements in a cost effective manner has put forward a challenging demand. For any fermentation based product, the most important thing is the availability of fermented product equal to that of market demand. Various microorganisms have been reported to produce an array of primary and secondary metabolites, but in a very low quantity. In order to meet the market demand, several high yielding techniques have been discovered in the past, and successfully implemented in various processes, like production of primary or secondary metabolites, biotransformation, oil extraction, and the like.

Medium optimization is still one of the most critically investigated phenomenon that is carried out before any large scale metabolite production and possess many challenges too. Before 1970s, media optimization was carried out by using classical methods, which were expensive, time consuming, and involving a great many experiments with compromised accuracy. Nevertheless, with the advent of modern mathematical/statistical techniques, media optimization has become more vibrant, effective, efficient, economical and robust in giving the results.

CO₂ concentration measurement in high sensitivity is important in terms of predicting process stage and biomass trend. CO₂ value is an important parameter in the growth, secondary metabolites biosynthesis and maintenance.

The nonlinear dynamics and multistage nature of the production stage can be monitored with high accuracy by CO₂ and additional control measurements. Unstructured models include cellular physiology information with a single biomass term without taken into consideration of the cellular activity. Structured models for secondary metabolites production include the effects of cell physiology on production by taking into account the physiology and differentiation of the cell change along the length of the hyphae and during fermentation.

There are a number of policies that are employed with the aim of optimizing production: (1) controlled sugar feeding rate to achieve a pre-decided growth pattern by controlling CO₂ concentration that can reflect the biomass growth rate at one preset value during growth phase and at another preset value during production phase by supplying a readily metabolizable sugar, e.g. glucose; (2) constant sugar feeding rate to reproduce a predefined growth pattern; (3) ramp increase or decrease (or exponential increase or decrease) of the sugar feeding rate to maintain a constant sugar concentration in the system during the production phase.

For an industrial fermentation process the process conditions, e.g. cultivation state, pH, agitation rate, dissolved oxygen concentration (dO₂), aeration etc. play a critical role because they effect the formation, concentration and yield of a particular fermentation end product thus effecting the overall process economics therefore it is important to consider the optimization process control in order to maximize the profits from fermentation process.

There are many challenges associated with optimization of fermentation processes. The optimization of different combinations and the sequence of process conditions, e.g., the trend and state of the cultivation, pH, agitation, aeration, and dO₂, and of medium components, e.g. nutrient addition such a carbon source and nitrogen source, need to be investigated, for a specific fermentation process, to determine the growth condition that produces the biomass with the physiological state best constituted for product formation. Additionally, the control of fermentation time will help in induction time (in the case of recombinant production for example), feeding control (in the case of fed-batch), the best time to harvest, etc.

A close-ended system is a system in which a fixed number and type of components and parameters are measured, e.g. pH, dO₂, CO₂, glucose, ammonia, agitation. This is the simplest strategy, but many different possible components/parameters which are not considered, could be beneficial in the medium. In an open-ended system any number and type of components/parameters are analyzed for optimization of fermentation process. The advantage of an open-ended system is that it makes no assumption of which components/parameters are best for the fermentation process. The ideal method would be to start with an open-ended system, select the best components/parameters for optimization of fermentation process then move to a close-ended system.

Optimization of production is required to maximize the end product yield. This can be achieved by using a wide range of techniques from classical “one-factor-at-a-time” to modern statistical and mathematical techniques, e.g. artificial neural network (ANN), genetic algorithm (GA) etc. Every technique comes with its own advantages and disadvantages, and despite drawbacks some techniques are applied to obtain best results.

“Biological mimicry” is a close-ended system for fermentation process optimization that is useful for optimization of various components of fermentation media. The method is based on the concept that a cell grows well in a medium that contains everything it needs in the right proportion (mass balance strategy). The medium is optimized based on elemental composition of microorganisms and growth yield. The limitation of this method is that measuring elemental composition of microorganisms is expensive, laborious and time consuming; moreover, the method does not consider the interactions of the components. However, this method gives an idea about the levels of different micro and macro elements required in the media for optimal growth of microorganisms.

Methods applied today for determining the culture trend and optimal growth conditions, e.g., optical density or live counts, need invasive sampling and therefore are prone to errors. Other online methods, such as pH or dO₂ measurements are not considered to be accurate to correlate with biomass.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for optimized industrial production using machine learning. The method comprises: creating a model defining dependencies among a plurality of parameters for an industrial production process, the plurality of parameters including a plurality of controlled parameters and a plurality of monitored parameters; training an agent via reinforcement learning based on iterative application of the model, wherein the agent is trained to determine new values for the plurality of controlled parameters based on current values of the plurality of monitored parameters in order to optimize the industrial production process with respect to at least one predetermined objective; and iteratively modifying, by the trained agent, current values of the plurality of controlled parameters in real-time during operation of the industrial production process.

Certain embodiments disclosed herein also include a non-transitory computer readable medium having stored thereon causing a processing circuitry to execute a process, the process comprising: creating a model defining dependencies among a plurality of parameters for an industrial production process, the plurality of parameters including a plurality of controlled parameters and a plurality of monitored parameters; training an agent via reinforcement learning based on iterative application of the model, wherein the agent is trained to determine new values for the plurality of controlled parameters based on current values of the plurality of monitored parameters in order to optimize the industrial production process with respect to at least one predetermined objective; and iteratively modifying, by the trained agent, current values of the plurality of controlled parameters in real-time during operation of the industrial production process.

Certain embodiments disclosed herein also include a system for optimized industrial production using machine learning. The system comprises: a processing circuitry; and a memory, the memory containing instructions that, when executed by the processing circuitry, configure the system to: create a model defining dependencies among a plurality of parameters for an industrial production process, the plurality of parameters including a plurality of controlled parameters and a plurality of monitored parameters; train an agent via reinforcement learning based on iterative application of the model, wherein the agent is trained to determine new values for the plurality of controlled parameters based on current values of the plurality of monitored parameters in order to optimize the industrial production process with respect to at least one predetermined objective; and iteratively modify, by the trained agent, current values of the plurality of controlled parameters in real-time during operation of the industrial production process.

The disclosed embodiments include a method, a system, and a controller for optimizing and controlling an industrial production process. In one or more embodiments, the method, system and controller allow for controlling feeding of input sources (e.g., carbon and nitrogen sources) in an industrial production process. In one or more embodiments, the method, system and controller allow for controlling physical parameters (e.g., agitation, pressure, and airflow) in an industrial production process. In one or more embodiments, a method includes constructing a mathematical model that mimics a dynamic behavior of a reactor. In one or more embodiments, methods include creating a trained agent using the mathematical model and a machine learning algorithm, the trained agent capable of providing controlled parameters for the production process. In one or more embodiments, a controller with the trained agent is used to process parameters monitored during the production process (herein referred to as “monitored parameters”) and provide as input to the reactor-controlled parameters applied during the process. In one or more embodiments, a controller including an agent trained using a mathematical model that mimics a behavior of an industrial process of a reactor is herein disclosed. In one or more embodiments, such controller includes a storage medium, a processor (e.g., a microprocessor) and the trained agent obtained using iterative training of the mathematical model.

The disclosed embodiments also include a method for optimizing and controlling parameters (e.g., nutrient feeding and physical parameters) in an example fermentation process.

The disclosed embodiments also include a system for optimizing and controlling the feeding of input sources and/or physical parameters in an industrial production process that is based on a model that mimics the behavior of the industrial production process.

The disclosed embodiments also include a system for optimizing and controlling the feeding of carbon and nitrogen sources in an example fermentation process.

In some embodiments, a method of automated control of an industrial reactor-based production process is provided including one or more of: collecting historical data about performance of a reactor, defining one or more of monitored parameters, defining one or more of controlled parameters, and defining a model including one or more equations mimicking a dynamic behavior of the reactor.

Some disclosed embodiments include a method of automated control of an industrial reactor-based production process, the method comprising: collecting data about previous performance of a production process of a reactor; defining a set of monitored parameters and a set of controlled parameters in the production process; defining a model comprising a set of equations mimicking a dynamic behavior of the process of a reactor, wherein in the model, changes in the monitored parameters are linked to changes in the controlled parameters; providing a machine learning computer program code; and creating a trained agent obtained using a machine learning code and the model, wherein the trained agent capable of making decisions regarding controlled parameters (actions) to be applied to the reactor based on monitored parameters detected during the production process.

Some disclosed embodiments include a method of automated control of an industrial reactor-based production process comprises the steps of: collecting data associated with performance of a production process of a reactor; defining a set of monitored parameters and a set of controlled parameters in the production process; defining a model comprising a set of equations mimicking a dynamic behavior of the process of a reactor, wherein in the model, changes in the monitored parameters are linked to changes in the controlled parameters; and creating a trained agent obtained by iterative machine learning training code and using the model, wherein the trained agent is capable of making decisions regarding controlled parameters to be applied to the reactor based on monitored parameters of the production process.

In some embodiments, by optimizing it is meant to refer to improving, or enhancing efficiency in obtaining one or more objectives (e.g., high product yield, short fermentation duration, low impurity value) of an industrial production process.

In some embodiments, a method of automated control of an industrial reactor-based production process further includes validating the model by comparing actual parameters obtained in actual production runs and/or in experimental production runs of the reactor with artificial predictive parameters obtained utilizing the model.

In some embodiments, a method of automated control of an industrial reactor-based production process further includes validating the model by comparing monitored parameters obtained in actual production runs and/or in experimental production runs of the reactor with artificial predictive monitored parameters obtained when providing controlled parameters (e.g., of actual production runs and/or in experimental production runs) and processing thereof using the model. In one or more embodiments, the validation of the model further includes determining a difference between the actual parameters and the artificial predictive parameters and determining that the difference (herein also refers to as “error”) does not exceed a predetermined threshold which may be an absolute value or a relative value. In some embodiments, a method of automated control of an industrial reactor-based production process further includes validating the model by: selecting initial input values, processing the initial input values by the model, resulting in a set of calculated predictive values for the monitored parameters, determining a difference between the calculated predictive values of the monitored parameters and respective values of the monitored parameters in the historical data, and further determining that the difference does not exceed a predefined threshold.

In some embodiments, a method of automated control of an industrial reactor-based production process comprises providing a code of a machine learning computer program, encoding operation of a state machine; wherein the state machine comprises a decision policy, comprising at least one changeable weight value that is linked to performance of an action (i.e., one or more controlled parameters) in response to at least one monitored parameter; defining a reward vector for providing rewards of consecutive episodes of the production process and obtaining consecutive decision policies with improved rewards; and iteratively training the state machine to thereby obtain a trained agent that can regulate the production process according to parameters monitored during the production process by one or more sensors within the reactor. In one or more embodiments, the iterative training of the state machine includes processing episodes of the production process and determining rewards for the episodes until maximal reward is yields thereby obtaining a trained agent that can maximize objectives of the production process of the reactor. In one or more embodiments, iterative training the state machine includes: applying a set of initial input values and processing thereof by the model, to thereby obtain a set of calculated predictive values for the monitored parameters, calculating a difference between a value of monitored parameters in the set of initial input values and a value of the monitored parameter in the set of calculated predictive values, determining that the difference calculated at the step of calculating is oriented within a direction of the reward vector, and altering the at least one changeable weight value in the decision policy, to maximize the specific change in the monitored parameter within the direction of the reward vector. In or more embodiments, rewards are determined according to one or more predefined objectives for the production process. In one or more embodiments, the objectives include one or more of a member selected from high product yield, short fermentation duration, low impurity value (for processes with impurity), product quality, process efficiency, and a combination thereof.

In some embodiments, a method of automated control of an industrial reactor-based production process is provided including storing the machine learning computer program code, with at least one altered weight value in the decision policy, resulting from the training, on a storage medium of a controller, connecting the controller to a local agent, configured for generating an executable code and/or real-time instructions for equipment-controllers of the reactor, according to the machine learning computer program code, with the at least one altered weight value of the decision policy, stored on the storage medium of the controller.

In some embodiments, a method of automated control of an industrial reactor-based production process is provided including operating the reactor in real-time by: consciously detecting values the monitored parameters; communicating the values of the monitored parameters to the controller; dynamically applying the controlled parameters in response to the monitored parameters.

In some embodiments, the reactor is selected from the group consisting of: fermenter, bioreactor, and chemical reactor.

In some embodiments, the previous operational periods of the reactor are selected from the group consisting of: routine production runs of the reactor and experiments configured specifically of the reactor.

In some embodiments, the defining of the model further comprises: selecting an equation comprising at least one constant; selecting a plurality of different values for the at least one constant; applying the initial input values to the equation with the plurality of different values for the at least one constant; determining which value from the plurality of different values for the at least one constant resulted to a minimal deference between the calculated predictive values of the monitored parameters and respective values of the monitored parameters in the historical data.

In some embodiments, the method comprises determining that the training has been performed to a sufficient extent.

In some embodiments, the defining of the model comprises calibrating the model, by selecting a set of values for constants of the equations.

In some embodiments, the altering of the at least one changeable weight value in the decision policy is performed after iteratively performing the sub-steps of applying, processing, calculating and determining at the step of training the state machine.

In some embodiments, the actions or controlled parameters applied to a reactor are dictated by preset tolerances. In some embodiments, the preset tolerances refer to such controlled values within a permitted range as determined by the specific production process.

In some embodiments, the method further comprises determining that the training has been performed to a sufficient extent, comprising at least one member selected from the group consisting of: determining absolute values of a reward value and determining changes in the reward value.

In some embodiments, the local agent is further configured for communicating values of the monitored parameters to the controller.

In some embodiments, an automated industrial reactor comprises a controller comprising: a storage medium comprising a standardized code of a machine learning computer program, encoding operation of a state machine, wherein the state machine comprises a decision policy, comprising at least one changeable weight value that is linked to performance an action in at least one controlled parameter, in response to in at least one monitored parameter, resulting from a previous training of the controller; a microprocessor configured for dynamically applying changes in controlled parameters in response to monitored parameters; a communication port configured for connecting the controller to a local agent; and a local agent configured for generating an executable code and/or real-time instructions for equipment-controllers of the reactor, according to a computer code, provided by the controller.

In some embodiments the training of the controller comprising: collecting historical data about performance of the reactor, during previous operational periods; defining a set of the monitored parameters and a set of the controlled parameters in the production process; defining a model comprising a set of equations, mimicking a dynamic behavior of the reactor, in the production process, wherein changes in the monitored parameters are linked to changes in the control parameters, and validating the model.

In some embodiments, construction of the controller comprises providing the standardized code of the machine learning computer program, encoding operation of the state machine; wherein the state machine comprises the decision policy, comprising the at least one changeable weight value that is linked to performance of an action in respect at least one in controlled parameter, in response to at least one monitored parameter; defining a specific change in at least one designated monitored parameter as a reward vector for the decision policy, according to a predefined objective; iteratively training the state machine by: applying a set of initial input values to the state machine, resulting in a set of output values for the controlled parameters; processing the set of output values of the controlled parameters by the model, resulting in a set of calculated predictive values for the monitored parameters; calculating a difference between a value of the at least one designated monitored parameter in the set of initial input values and a value of the at least one designated monitored parameter in the set of calculated predictive values; determining that the difference calculated at the step of calculating is oriented within a direction of the reward vector; altering the at least one changeable weight value in the decision policy, to maximize the specific change in the at least one designated monitored parameter within the direction of the reward vector.

In some embodiments, the reactor is selected from the group consisting of: fermenter, bioreactor, and chemical reactor.

Some disclosed embodiments include a controller for controlling parameters of an industrial production process, the controller comprising: a storage media; a microprocessor; and a communication port configured for connecting the controller to a local agent of a reactor of a production process, wherein the local agent configured to transmit data regarding monitored parameters of the production process to the controller and data regarding controlled parameters to be applied to the reactor; wherein the controller comprising a trained agent capable of dynamically applying changes in controlled parameters in response to monitored parameters, the trained agent obtained from training an agent of a mathematical model constructed for the production process of the reactor that mimics the behavior of the reactor.

In one or more embodiments, the local agent configured for generating an executable code and/or real-time instructions for reactor or to equipment-controllers of the reactor, according to the trained agent provided by the controller.

In one or more embodiments, the mathematical model constructed by: collecting historical data about performance of the reactor, during previous operations thereof; defining a set of the monitored parameters and a set of the controlled parameters in the production process; and defining a model comprising a set of equations, mimicking a dynamic behavior of the reactor, in the production process, wherein changes in the monitored parameters are linked to changes in the control parameters.

In one or more embodiments, the model is validated by: selecting initial input values for the controlled parameters and for the monitored parameters; processing the initial input values by the model, resulting in a set of calculated predictive values for the monitored parameters; determining a difference between the calculated predictive values of the monitored parameters and respective values of the monitored parameters in the historical data; and further determining that the difference does not exceed a predefined threshold.

In one or more embodiments, training the agent comprises: providing a real time executable code of a machine learning based computer program encoding operations of a state machine; wherein the state machine comprises an, comprising at least one changeable weight value that is linked to controlled parameter provided in response to monitored parameters; applying consecutive episodes of the process of the reactor; calculating rewards for the episodes; wherein the rewards defined according to a predefined objective for the production process; updating the agent based on improved rewards and by altering the at least one changeable weight value in the agent to maximize the rewards; and determining a trained agent receiving a maximal reward of the episodes.

In one or more embodiments, the predefined objective selected from high product yield, short fermentation duration, product quality, process efficiency, low impurity value and a combination thereof.

Some disclosed embodiments include an automated industrial production system for an automated production process, the system comprising: an industrial production reactor, a controller comprising a storage media, a microprocessor, and a communication port configured for connecting the controller to a local agent; and a local agent configured to transmit data regarding monitored parameters of the production process to the controller and data regarding controlled parameters to be applied to the reactor; wherein the controller comprising a trained agent capable of dynamically applying changes in the controlled parameters in response to monitored parameters, the trained agent obtained by iterative training using machine learning computer program and a mathematical model constructed for the production process of the reactor that mimics the behavior of the reactor.

The term “model” as used herein refer to a mathematical set of equations that mimics the dynamic behavior of a specific industrial production process. The term model as referred to herein is often referred to in reinforcement learning field as “environment”.

The term “monitored parameters” are parameters whose values give information about the status of the industrial production process, e.g. CO₂ concentration, dO₂, pH, carbon source concentration, nitrogen source concentration in an example fermentation process, the monitored parameters are measured by sensors in or at the fermenter.

The term “controlled parameters” are values of parameters that are input to the industrial production process, such as in the example of a fermenter to control the fermentation process, e.g. the rate of feeding a carbon or a nitrogen source, or to control operation of the fermenter, e.g. agitation or aeration rates and temperature control.

The term “state machine” is an operational computer program code, encoding for storing the status of monitored parameters at a given time, calculating the status changes of monitored parameters and determining the resulting output for the controlled parameters implementing the changes. The monitored parameters may, in certain embodiments, include objectives of the herein disclosed embodiments such as high product yield, short fermentation duration, and low impurity value.

The term “controller” and/or “services” is a computational device the microprocessor of which executes the operational computer program code or trained agent encoding the operation of the state machine and the storage media of which typically stores the operational computer program code of the state machine.

The term “system state” is a vector of the selected monitored parameters for the operation of a state machine of the controller at a specific timestamp, the system state can also contain past values or statistical procedures carried out on the values.

The term “agent” is a utility, i.e. software algorithm, designed to determine the action for each system state that will improve the performance of the process in terms of the goal function selected, in the controller.

The term “actions” is setting the values of the controlled parameters as a result of the system state, which can result in a change in the controlled parameters.

The term “episode” is a complete simulation of the modeled process, conducted during the training stage; the controlled parameters during this run are determined using the controller based on past episodes. After an episode, the controller is updated using an agent's decision policy obtained for an updated/maximized “reward”.

The term “reward” is a score function, designed specifically for each process, which evaluates the decisions that the controller made during an episode. After the reward value calculation, it is used to update the controller or an agent thereof for future episodes. The reward could be based on the parameters the controller aims to improve, e.g., productivity, impurity, production time etc.; the reward can be determined from different score functions for different times during the process.

The term “weight values” as referred to herein is often referred to in machine learning as “weights”, which is a value that is altered as a result of the reward.

The term “local agent” is a computer program that mediates between the chemical or biological reactor, such as a fermenter, and the agent or any hardware component which receives the data of monitored parameters from the sensors, sends them to the controller, receives back controlled parameters values and sends them to the controllers of the industrial equipment, such as PLCs.

The term “trained agent” as used herein refers to an agent or a computer code iteratively trained by machine learning techniques. The trained agent may be determined with respect to a decision policy with the maximal/best yielded reward obtained by the iterative training.

Whenever the terms “server”, “agent”, “system” or “module” is used herein, it should be construed as a computer program, including any portion or alternative thereof, e.g. script, command, application programing interface (API), graphical user interface (GUI), etc., and/or computational hardware components, such as logic devices and application integrated circuits, computer storage media, computer micro-processors and random access memory (RAM), a display, input devices and networking terminals, including configurations, assemblies or sub-assemblies thereof, as well as any combination of the former with the latter.

The term “storage” as referred to herein is to be construed as including one or more of volatile or non-volatile memory, hard drives, flash storage devices and/or optical storage devices, e.g. CDs, DVDs, etc.

The term “computer-readable media” as referred to herein can include transitory and non-transitory computer-readable instructions, whereas the term “computer-readable storage media” includes only non-transitory readable storage media and excludes any transitory instructions or signals.

The terms “computer-readable media” and “computer-readable storage media” encompass only a computer-readable media that can be considered a manufacture (i.e., article of manufacture) or a machine. Computer-readable storage media includes “computer-readable storage devices”. Examples of computer-readable storage devices include volatile storage media, such as RAM, and non-volatile storage media, such as hard drives, optical discs, and flash memory, among others.

The term “integrated” shall be construed inter alia as operable on the same machine and/or executed by the same computer program. Depending on the actual deployment of the method, its implementation and topology, integration of agents and/or integration into modules as well as the terms “transfer”, “relaying”, “transmitting”, “forwarding”, “retrieving”, “accessing”, “pushed” or similar refer to any interaction between agents via methods inter alia including: function calling, Application Programming Interface (API), Inter-Process Communication (IPC), Remote Procedure Call (RPC) and/or communicating using of any standard or proprietary protocol, such as SMTP, IMAP, MAPI, OMA-IMPS, OMA-PAG, OMA-MWG, SIP/SIMPLE, XMPP, SMPP.

The term “network”, as referred to herein, should be understood as encompassing any type of computer and/or data network, in a non-limiting manner including one or more intranets, extranets, local area networks (LAN), wide area networks (WAN), wireless networks (WIFI), the Internet, including the world wide web, and/or other arrangements for enabling communication between the computing devices, whether in real time or otherwise, e.g., via time shifting, cashing, batch processing, etc.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 schematically shows CO₂ concentration (curve A), biomass concentration (curve B), and carbon source concentration (curve C) as functions of time;

FIG. 2 is a graph showing CO₂ concentration as a function of time for an actual production run that shows the effect of lack of nitrogen source on the CO₂ concentration during the production phase of an example fermentation process emphasizing how a non-carbon source can affect and be described by the CO₂ dynamic;

FIG. 3 is a graph showing how a method for controlling the process can save time in an example fermentation process;

FIG. 4 schematically shows a closed loop system for optimized feeding of the carbon source and the nitrogen source feeding in an example fermentation process;

FIG. 5 shows a comparison of yield of fed-batch produced product, for production runs carried out by following the protocol previously used with yield obtained by using the system;

FIG. 6 shows graphs of CO₂ concentration and carbon source feeding as functions of time during a production run for a secondary derivative in which the carbon source was fed according to the standard protocol followed for production of the product;

FIG. 7 shows graphs of CO₂ concentration and carbon source feeding as functions of time during a production run for a secondary derivative in which the carbon source was fed according to the method; and

FIG. 8 schematically shows the reinforcement learning iterative training phase of an embodiment of the method;

FIG. 9 schematically shows control of the fermenter by the trained agent during live production runs;

FIG. 10 schematically shows an embodiment of a closed loop system configured for carrying out an embodiment of the method for optimizing values of the nutrient feeding and physical parameters in an example fermentation process;

FIG. 11 is a schematic diagram of an example computing environment according to some embodiments;

FIG. 12 shows graphs of the predicted and measured biomass concentration (top left graph), dissolved oxygen (bottom left graph), carbon source concentration (top right graph), and desired product concentration (bottom right graph) as measured during the validation phase of the model according to some embodiments;

FIG. 13 shows a graph displaying the learning process of a reinforcement learning algorithm according to some embodiments;

FIG. 14 is an example code of a reinforced learning machine according to some embodiments.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

Some disclosed embodiments relate to controlling particularly an example industrial production process by a reactor controller that regulates controlled parameters of the reactor continually during the process. In one or more embodiments, the industrial production process inter alia includes research and development processes, processes of pilot facilities, processes of demo facilities, fermentation processes, bio-reactor processes, and chemical processes. In one or more embodiments, the reactor includes various vessel processes including, but not limited to a bio-reactor, a chemical reactor and a fermenter. In one or more embodiments, the controlled parameters, inter alia include the amounts of nutrient sources to feed the process, the timing of the feedings, and/or physical parameters such as agitation, aeration rates and temperature control.

In an embodiment, the method generally comprises several phases for obtaining an agent trained using a mathematical model that simulates the actual production process of a reactor.

A controller as herein disclosed includes a trained agent or the controller may be trained to obtain a trained agent that can maximize objectives of the production process.

Thus, some embodiments include methods for regulating a production process of a reactor.

Some embodiments include a controller with a trained agent or an agent that can be trained based on a model that mimics the production process of a reactor.

Some embodiments include systems with a controller, a local agent and a reactor.

In one or more embodiments, the method includes a phase in which a mathematical model is constructed; a learning/optimization phase, and a production, i.e. “real time”, phase. These phases of the method are unique for each industrial production or fermentation process and the exact steps required to carry them out must be determined specifically for each particular process. In one or more embodiments, a model constructed for a particular process is optimized during the learning/optimization phase and the optimized model is then used to obtain a trained agent. In one or more embodiments an agent is trained during the learning/optimization phase and the optimized agent or trained agent is used to automatically control and optimize production runs of an example fermentation process.

The mathematical model is optionally a set of equations, optionally differential equations collectively comprising parameters that describe different aspects of the specific example production process being controlled and optimized. The equations may be based on the academic literature, past data collected on the process, and the results of specifically designed experiments.

The use of differential equations as a basis for a model representing growth and activity of microorganisms is known and used in research and several industries for a better understanding of the interactions of different elements in the process, and in some cases as a basis for improving current protocols using knowledge gained from the model. Other control mechanisms such as pH control or dO₂ control, used in the example fermentation processes, calculate input values using a strict set of rules, wherein each variable measured could usually influence changes in one input value controlled. For example, pH can be titrated to adjust a specific set point and dissolved oxygen (dO₂ control) can be used as a set point to control fermentation parameters such as temperature and pressure (agitation/airflow adjustment).

The disclosed method, using a model of the specific process, integrates all live measured data, along with data from past measurements of the process for a full image of the current conditions of the fermenter. The controller integrates machine learning and optimization methods to find the best possible input or controlled parameters to the fermentation vessel at each time during the process, e.g. quantity of C and N source, temperature, agitation or aeration rate.

Optionally, machine learning and optimization methods as herein disclosed make use of past and/or real time data collected from a reactor to find the best possible controlled parameters to the fermentation vessel.

A model can be created for different products produced by the example fermentation process. In a specific example, the production of biomass, i.e. microbial cells or biomass is sometimes the intended product of an example fermentation process. Non limiting examples of such processes include production of single cell protein, baker's yeast, lactobacillus, E. coli, and other, extracellular primary metabolites and secondary metabolites. Some examples of primary metabolites are ethanol, citric acid, glutamic acid, lysine, vitamins and polysaccharides. Some examples of secondary metabolites are penicillin, cyclosporin A, gibberellin, and lovastatin. These compounds are of obvious value to humans wishing to prevent the growth of bacteria, either as fed-batch produced products or as antiseptics (such as gramicidin S) or fungicides, such as griseofulvin, which are also produced as secondary metabolites. Typically, secondary metabolites are not produced in the presence of glucose or other carbon sources which would encourage growth and like primary metabolites are released into the surrounding medium without rupture of the cell membrane. Of primary interest among the intracellular components are microbial enzymes: catalase, amylase, protease, pectinase, glucose isomerase, cellulase, hemicellulase, lipase, lactase, streptokinase and many others. Examples of recombinant proteins that are produced in fermentation processes include insulin, hepatitis B vaccine, interferon, granulocyte colony-stimulating factor, and streptokinase.

A specific example of creation of a model for secondary metabolites in a fed-batch fermentation process follows:

In this model, t represents the time and the model is updated with a time differential of dt.

The biomass trend is given by equation (1):

X(t+1)=X(t)+dt(X(t)(μ(t−K_{d})) (1)

wherein: X(t) is the biomass concentration in the fermenter at time t, K_{d} is the death factor constant of the cells, and μ(t) is the growth rate of the cells at time t, X(t+1) is the value of X(t) one minute after t, and μ(t) is given by equation (2):

$$μ ( t ) = {μ}_{x} ( \frac{S ( t )}{{K}_{x} + S ( t )} ) ( \frac{C L ( t )}{C L ( t ) + {K}_{o x}} ) ( \frac{A ( t )}{A ( t ) + {K}_{x a}} ) \qquad ( 2 )$$

wherein: μₓ is the maximal growth rate constant of the cells, Kₓ is the carbon source limitation constant for growth, Kₒₓ is the oxygen limitation constant for growth, S(t) is the carbon source concentration in the fermenter at time t, and CL(t) is the dissolved oxygen concentration at time t, A(t) is the nitrogen source concentration in the fermenter at time t, and Kₓₐ is the nitrogen source limitation constant for growth.

The production trend is given by equation (3):

P(t+1)=P(t)+dt(μₚₚ(t)X(t)−KP(t)) (3)

wherein: P(t) is the product concentration in the fermenter, K is the product hydrolysis rate constant, and μₚₚ(t) is the production rate at time t, μₚₚ(t) is given by equation (4):

$${μ}_{p p} ( t ) = {μ}_{p} ( \frac{A ( t )}{{K}_{p} + A ( t )} ) ( \frac{C L ( t )}{C L ( t ) + {K}_{o p}} ) ( \frac{S ( t )}{{K}_{I} + S ( t )} ) \qquad ( 4 )$$

wherein, μₚ is the maximal production rate constant, Kₚ is the production inhibition constant for ammonia, Kₒₚ is the production inhibition constant for dissolved oxygen, and K_{I} is the inhibition constant for dextrose.

The carbon source is used for cell growth, production and maintenance of the fermentation process. The amount of carbon source in the fermentation vessel decreases with time and can be increased by feeding during the process. The carbon source trend is given by equation (5):

$$S ( t + 1 ) = S ( t ) + d t [ X ( t ) ( - \frac{μ ( t )}{{Y}_{x / s}} - {m}_{x} - \frac{{μ}_{p p} ( t )}{{Y}_{p / s}} ) + \frac{{S}_{i n} ( t )}{Vessel volume} ] \qquad ( 5 )$$

wherein, S(t) is the carbon source concentration in the fermenter at time t, Y_{x/s} is the growth yield constant for the carbon source, Y_{p/s} is the production yield constant for the carbon source, mₓ is the maintenance constant of the carbon source, and Sᵢₙ is the carbon source feeding value.

Nitrogen is needed for production. The amount of nitrogen can be increased when needed by feeding. The nitrogen source trend is given by equation (6):

$$A ( t + 1 ) = A ( t ) + d t * ( X ( t ) ( - \frac{{μ}_{p p ( t )}}{{Y}_{p / a}} - \frac{μ ( t )}{{Y}_{p / s}} + \frac{{A}_{i n} ( t )}{Vessel Volume} ) \qquad ( 6 )$$

wherein: A(t) is the nitrogen source concentration in the fermenter at time t, Y_{p/a}-Growth Yield constant for the nitrogen source, μₚₚ is the specific fed-batch produced product production rate, and Aᵢₙ is the Nitrogen source feeding value.

The dissolved oxygen trend, showing the uptake of oxygen by the cells, is given by equation (7):

$$CL ( t + 1 ) = CL ( t ) + d t [ X ( t ) ( \frac{- μ ( t )}{{Y}_{x / o}} - {m}_{o} - \frac{{μ}_{p p} ( t )}{{Y}_{p / o}} ) + {K}_{l a} ( C {L}^{*} - C L ( t ) ) ] \qquad ( 7 )$$

wherein: CL(t) is the dissolved oxygen level in the fermenter at time t, CL* is the maximal dissolved oxygen concentration, Y_{x/o} is the growth yield constant for dissolved oxygen, Y_{p/o} is the production yield constant for dissolved oxygen, mₒ is the maintenance constant of dissolved oxygen, and Kₗₐ is the oxygen insertion constant.

Each process has its specific properties and different fermentation processes will have different values of these properties in the above equations as well as a different set of equations. Properties could be added or removed for example in cases of inducers, a second carbon/nitrogen source, or a second product. The equations could change as well due to different kinetics and relations between variables. Different processes could be a result of different fed-batch produced products, different organism (bacterium or fungi), or different fermentation procedures.

In view of the above, one or more of the herein disclosed systems and methods include one or more of the following stages:

A Model Creation Phase

Stage 1—The mathematical model is built, i.e., theoretical equations that describe various aspects of the process are chosen. The equations that are selected collectively comprise various, optionally all parameters that describe different aspects of the specific fermentation process being investigated.

Stage 2—Data relating to the values of the parameters in the equations is gathered from production runs and observation trials. In this stage, data is collected from as many real production runs and from variations to the real runs that are performed during the observation trials.

Stage 3—The data of controlled parameters collected in stage 2 is inserted into the equations, which are simultaneously solved to obtain predictive output of predictive monitored parameters.

Stage 4—a comparison is then performed between real time output of monitored parameters and the predictive values, and a base model that best fits the production process is chosen.

An Agent Creation Phase

Stage 5—Machine learning techniques and the model are used to create a trained agent that is used for future production runs.

In one or more embodiments, the above stages 1 to 5 are conducted offline, i.e., when not connected to an actual real time production process but rather performed artificially using the model that simulates the actual production process of the reactor.

The following is an example explanation of Carbon and Nitrogen source feeding based on the disclosed model.

One of the factors leading to less than optimal yields and profitability of fermentation processes as carried out today in industries such as the pharmaceutical industry is that material, such as the carbon source that is necessary to promote cell growth and production is added to the fermentation vessel in predetermined quantities at fixed times that have been determined by trial and error during an initial running-in period of the process before commercial production of a new product begins. The nitrogen source is added in real time by titrating the pH during the fermentation process.

Based on their conviction that yields and profitability can be significantly increased by feeding the carbon and nitrogen sources only in the amount and at the time that is needed, the inventors have developed a method and a controller for using the model derived as described herein to dynamically provide optimal values of selected controlled parameters to a reactor. The controller as herein disclosed receives real-time data of monitored parameters measured by sensors attached to the fermenter and instructs a local agent and/or equipment controller devices of the reactor (e.g., a pump, an agitation device, a nutrient feeding device, etc.,) to adjust the controlled parameters of the reactor in order to optimize the process with respect to quantity and purity of the final product and the overall cost. For example, the value of the pH will be controlled by addition of nitrogen source, e.g. ammonia; dO₂ will be controlled by adjusting pressure or temperature; CO₂ concentration will be controlled by addition of carbon source, e.g. glucose, by agitation, or by adjusting the values of other parameters that will affect the biomass trend. Specifically, since overfeeding can cause toxicity and underfeeding will cause increased CO₂ levels with increased biomass growth and no production. Both situations are described in the model, which is optimized to provide the fermentation controller with controlled parameters values that will prevent either of them from occurring.

Nitrogen source along with carbon source are two substrates necessary for an example fermentation process. During the growth phase, the carbon source is used in a “Krebs cycle” (glycolysis cycle) and CO₂ is released. During the production phase, cell growth is reduced and equilibrium between carbon and nitrogen source is required for high yield production. Lack of carbon source concentration will cause reduced production, cell maintenance and cell growth (biomass), and therefore will cause a decrease in CO₂ levels. On the other hand, lack of nitrogen source concentration needed for product creation will shift the culture back to the growth phase, meaning that the carbon source will be used for glycolysis, and the CO₂ concentration will increase. These traits are modeled, as described herein above, by adjusting the equations, finding the relevant values of the properties, and optimized for an efficient and productive process.

During the rapid growth rate of cells, a minimal medium containing, e.g. glucose, is required as the sole source of carbon. During the growth phase metabolism of glucose to smaller molecules (e.g., CO2, ethanol, or acetic acid) can generate the ATP necessary for energy-requiring activities of the cells. The sole nitrogen source in a minimal medium can be ammonium (NH4+), from which the cells can synthesize all the necessary amino acids and other nitrogen-containing metabolites.

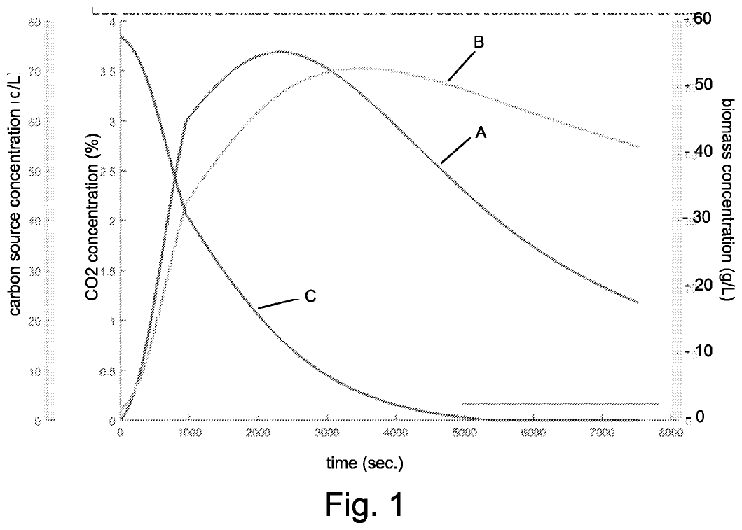

FIG. 1 schematically shows CO₂ concentration (curve A), biomass concentration (curve B), and carbon source concentration (curve C) as functions of time for a typical fermentation process. High correlation between CO₂ and biomass concentrations, especially during the growth stage, is visible. This is with negative correlation between the CO₂ concentration and the carbon source concentration, meaning that the carbon source is being used for the biomass growth. During the production phase, biomass growth rate decreases and the resources are used for the second metabolite formation as well.

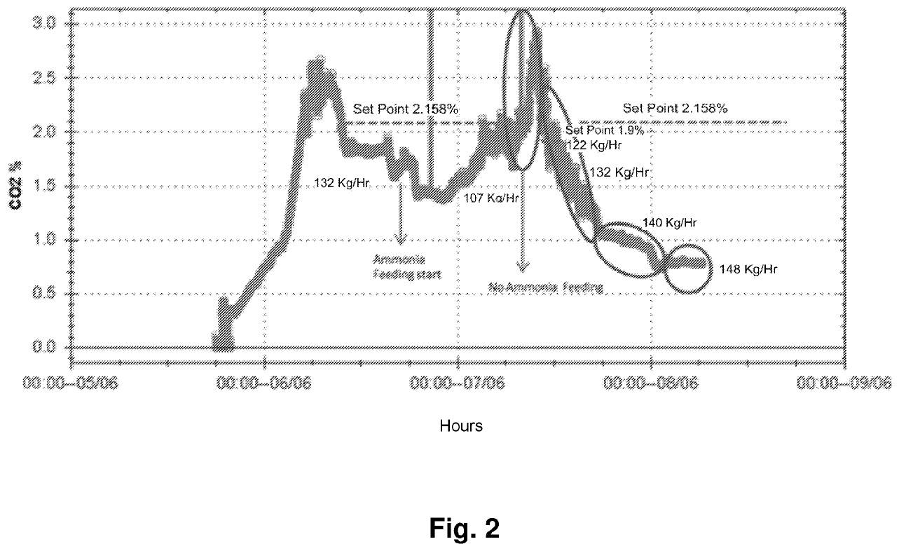

FIG. 2 is a graph showing CO₂ concentration as a function of time for an actual production run that shows the effect of lack of nitrogen source on the CO₂ concentration during the production phase of an example fermentation process. In the figure the dashed line is the set point for the CO₂ concentration with the values of the set point written above the line. The rate at which the carbon source (sugar) is fed at various stages into the fermenter is written next to the curve. Carbon source is fed starting after about 5.75 hours in equal doses every minute, e.g. during the growth phase 2.2 kg of sugar are added each minute. Feeding with ammonia begins at about 6.75 hours (indicated by a downward pointing arrow). The amount and timing of ammonia feeding was controlled to keep the pH within predetermined upper and lower limits. Between 7.25 and 7.5 hours, during the period marked by the ellipse with the vertically pointing arrow at its bottom, the ammonia supply was exhausted, and no ammonia was fed until a new supply was prepared. During this time it can be seen how the process shifted from the production to the growth phase accompanied by a rapid rise in CO₂ concentration.

As discussed above and shown schematically in FIG. 1, during the growth phase the biomass state is closely coordinated with the state of the CO₂ concentration; and, as shown in FIG. 2, during the production stage, the CO₂ concentration is influenced by both the nitrogen and the carbon source concentrations. The CO₂ concentration at any time during the process depends on the metabolism of cells in the fermenter, which in turn depends directly on the feeding rate of the carbon and nitrogen sources. These facts indicate that CO₂ concentration can be useful in feeding control of both C and N; and, in view of them, the inventors have developed a closed loop system that uses mathematical model of the process derived by the method described herein above to control the level of the CO₂ concentration as a function of time, thereby providing a means of controlling the feeding of the carbon and nitrogen sources in an example fermentation process in accordance with the requirements of the process.

Some disclosed embodiments include a method of controlling an example fermentation process by a fermenter controller that regulates controlled parameters of the fermenter. Steps of such a method may include construction of a digital model that mimics the behavior of the fermentation process, processing input controlled parameter values of actual real time production runs by the model and obtaining predictive values of the monitored parameters, and comparing the values of these parameters to monitored values obtained received in real time during production runs from sensors at the fermenter. Comparison of the output from the model to the real time output data of real production runs is then used to obtain a model that mostly fits or mimics the actual behavior of the process. The input values calculated by the model may include controlled parameters obtained by real production processes. A trained agent based on the model obtained utilizing machine learning technique is then used to instruct the fermenter controller to adjust controlled parameters relating to the operation of the fermenter.

The controller that provides the input to the fermenter controller devices is based on biological mimicry model. The model is utilized for an example fermentation process for production of a specific product. The model contains various, optionally all parameters of the fermenter's operation and its contents that are related to the fermentation process.

In an optional embodiment, data is gathered from actual and experimental production runs. The data is inserted into the model and various algorithms are employed to determine a set of values for all parameters that best fits the data. Machine learning using input from subsequent production runs is used to optimize and continually update the model.

The model is useful for production in fermentation processes. Creating the model for a specific process comprises two phases: in the first phase experimental data on the fermentation process is gathered from which a digital model of the fermentation process is generated; second, by implementing optimization and machine learning methods, productivity increase is achieved.

In the first phase of creating the model, a base model is generated, which simulates the different interactions of the conditions inside the real fermenter for a specific fermentation process. Specifically, a mathematical model is created such that monitored parameters are linked to controlled parameters in a manner where changes in controlled parameters result in changes in the controlled parameters. The model may be based on a set of partial differential equations, representing the condition of the culture inside the fermenter at any time, while relations between variables (i.e., monitored and controlled parameters) are integrated in the equations.

The base model receives initial conditions, as well as input data from measurements of properties which effect the culture's state, e.g., carbon source/ammonia feeding values, agitation and air flow, along the simulated fermentation, and calculates the variable's values, e.g. carbon dioxide concentration, biomass concentration, carbon source/ammonia concentration, product concentration, and dissolved oxygen concentration—all as functions of time along the duration of the fermentation process.

After understanding the mathematical equations representing the process, the next step is approximation of the mathematical model to the physical process by finding accurate values for the properties in these equations. This approximation/validation is done using data collected from actual production batches, and from R&D experimental batches specially designed for understanding of certain aspects of the model. These experiments may optionally include specific properties which may be strictly controlled creating a different environment than the usual production state.

These measurements contain both the input data, such as feeding quantities and physical measurements (temperature, weight, airflow, agitation frequency and more) and the various variable values of the properties at all times. Feeding and physical measurements data are loaded into the model, which calculates the values of the properties. Then, the accuracy of the model is measured by comparing measurements of the real batch's properties to the output of the model. In this way several models are derived. An optimized model that represents the actual process with the highest precision is chosen where in such model the difference between real batch's measurements and output or predictive measurements of the model is minimal or does not exceed a specific or predefined threshold. Optionally, a performance score through a specially designed goal/objective function, where the most accurate model has the lowest goal/objective function score. Non-limited examples of goals/objectives include product yield, short fermentation duration, product quality, process efficiency, low impurity value and a combination thereof. Finally, various optimization methods, fitted for this purpose are activated, adjusting the values of the properties for an optimized model, with the lowest possible goal function score, that represents the actual process with the highest precision.

The model is optimized for a specific fermentation process for production of a specific product, for example, production of secondary derivative, enzyme or a specific fed-batch produced product, by a specific strain, and the optimization is done using data from real fermentation processes where all the values of the properties are measured and saved. This data is used for obtaining the values in the differential equations of the model that match the relevant process so that the digital fermenter created will behave in the same way as the physical fermenter. For that reason, the more data that is collected, with more diversity, a better, more accurate model can be created. In fermentation processes, particularly in secondary metabolites production, values of the properties of the process are closely related to medium composition and feed composition. When constructing a model for a specific process, it is essential to assess the adequacy of experiments for their validity and appropriateness of the kinetic and operation properties to use them with different medium and strain conditions. The model's fitting process is done using optimization methods that use the input data received for the construction of the simulated model to minimize the differences between the simulated values and the values measured in the actual fermentation process conducted.

The following is an example explanation of process enhancement using the disclosed model.

The model obtained in the first phase serves as a digital simulation of the real fermentation process. Therefore, after creation and validation of the model, it may be updated by machine learning techniques to obtain an optimized digital clone that is incorporated into a controller and to a local agent that can instruct dedicated instrumentation of the reactor to apply selected controlled parameters to a reactor based on parameters monitored by one or more sensors of the reactor. In one or more embodiments, machine learning and optimization methods take one or more of the following three final objectives into consideration: (1) high product yield, (2) short fermentation duration, (3) low impurity value (for processes with impurity). Achieving these objectives increases profitability by: creating more product; saving usage time of the fermenter, which can be used for more batches of the same process or of other processes; and saving resources used for purifying the product.

Different approaches may be used for calculation of the best possible controlled parameters:

(1) Creating an optimized digital fermentation process using optimization methods based on the created model. In addition, interactions between monitored parameters and controlled parameters are deduced from the model. The optimized digital process that is created is used as a template for model, which will aim for the preferred conditions at any time along the process through interactions knowledge obtained. An example of how this process works is to use a controller that uses a proportional-integral-derivative (PID) mechanism for each of the monitored parameters. A set point and bias are calculated for each of the parameters. Then close loop feeding control is achieved by the PID calculation for the specific bias and set point values that were calculated by using the model and the output rate is given by the PID controller.

(2) Dividing the process into phases (such as growth phase, production phase with abundant/lack of carbon source concentration in solution, stationary phase due to lack of necessary substrate etc.) that will be identified using supervised machine learning methods with measured data as features and past data as training. Each phase will have different preferred conditions that the processor will aim to at any time along the process through interactions knowledge obtained from the model.

(3) Activation of the model with various controlled parameters values every specified time period (usually according to measurements frequency), using data from current and past measurements, where initial conditions are set to be the current state of the fermenter. The results of the model will be treated by optimization methods in order to find the input values which leads to the best conditions in the future. This approach can be implemented after deciding the current process phase, using machine learning methods in a manner similar to the 2^{nd} approach.

All of these approaches reflect a model that uses all measured data as a base for the input values controlled through utilization of sophisticated algorithms; as a result, the method described herein is capable of achieving better profitability improvements compared to control mechanisms currently used in the art.

In outline the two phases of the method of generating the model described herein can be described as comprising the following six stages:

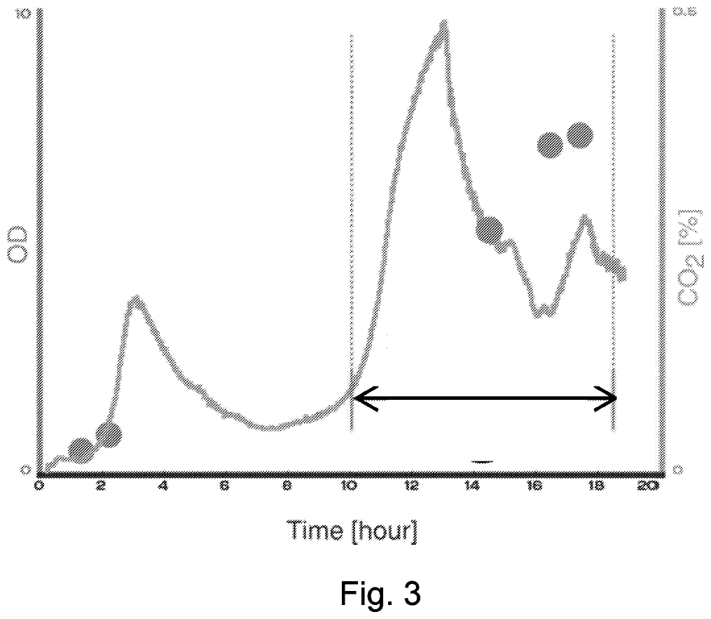

FIG. 3 is a graph showing how the method for controlling the process can save time in an example fermentation process during recombinant protein production. The figure shows the CO₂ concentration as a function of time and five measurements of optical density (OD) made during a process for production of a recombinant protein. In the method presently used by the operators of the system the measurements of the OD are used to determine when to add inducer to the process and start the recombinant protein production. At this stage, when inducer was added to the culture media, CO₂ concentration dramatically decreased, thus emphasizing the cells' state by terminating their replication stage/“birthing”/CO₂ release, and initiating use of their energy for the recombinant protein production. According to this method when the increase in OD is observed at 18 hours and after increased OD that show cell growth initiates again after the production stage has been accomplished, the process is stopped. According to the an embodiment, the CO₂ concentration is continuously monitored and, according to the understanding that the rapid rise of CO₂ at 10 hours is the result of rapid cell growth caused by end of production stage, the process would be stopped at 10 hours saving approximately nine hours.

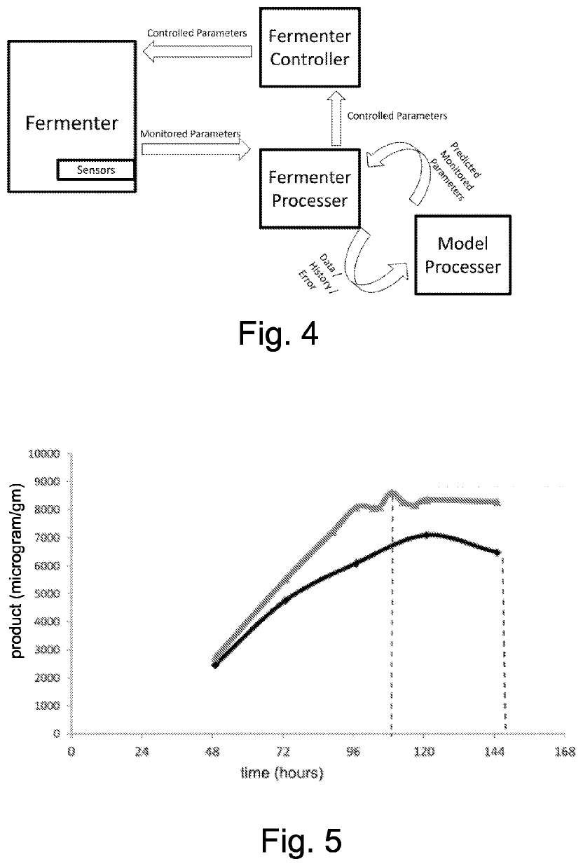

FIG. 4 schematically shows a closed loop system for optimized values of the nutrient feeding and physical parameters in an example fermentation process.

A fermenter processor, which may constitute part of the fermenter controller, receives, from sensors in a fermenter in which an example fermentation process is being carried out, instantaneous values of a set of monitored parameters as a function of time during the entire time of the process. The monitored parameters include inter alia: CO₂ concentration, nitrogen source and carbon source concentration, dO₂, pH, temperature, air flow, and agitation. The fermenter processor may optionally receive from the model predicted values of the monitored parameters. This is particularly relevant in cases where one or more of the monitored parameters are cannot be detected and/or assessed by the sensors of the fermenter. Software in the fermenter processor comprises a trained agent integrating the model updated using algorithms of machine learning and optimization methods to thereby generate controlled parameters. The values of the controlled parameters are sent in real time to the fermenter controller equipment in order to control operation of the fermenter. The controlled parameters may be the feeding of the nutrient sources, and physical parameters like agitation and aeration. For example, the instructions could be to change the agitation rate or to add a specified amount of carbon or nitrogen source.

In an optional embodiment, software in the fermenter processor comprises algorithms that use machine learning and optimization methods to generate controlled parameters that are based, inter alia, on various options of predicted values of the monitored parameters received from the model processer, after the model processer was activated with various options of controller parameters values. The values of the controlled parameters are sent in real time to the fermenter controller in order to control operation of the fermenter. The controlled parameters are the feeding of the nutrient sources, and physical parameters like agitation and aeration. For example, the instructions could be to change the agitation rate or to add a specified amount of carbon or nitrogen source. Data which might include the values as a function of time of the monitored or controlled parameters and the difference between the predicted and measured monitored parameters are sent in real time from the fermenter processor to the model processor, which uses the data to update the current model and optimize it generating a new model and to predict updated values of the monitored parameters, which in turn are sent back to the fermenter processer in real time.

It is noted that FIG. 4 depicts fermenter processor and the fermenter controller as separate physical entities, embodiments may comprise only a single controller with a processor containing software configured to carry out the functions described above.

In one or more embodiments, the criteria in the algorithms in software in the fermenter controller that are used to determine time and quantity of carbon source and nitrogen source feeding are based on the values and trends of the following parameters:

μₚₚ(t) presented in equations numbers 4 and 6, is the parameter that describes the production and makes the connection between the N source and the model, this parameters basically shows that the production rate is effected from substrate utilization and ammonia uptake by the cells;

μ(t) presented in equation number 2, describes the specific growth rate which is directly connected to the CO₂ and is influenced, in the growth stage, by both increases and decreases in the levels of carbon source and, during the production stage, by increases and decreases of both C and N.

Equation 2 describes the growth with dependence on both carbon S(t), oxygen concentration CL(t), and Ammonia A(t)

Although any commercially available CO₂ and pH sensor can be used in the system, for a CO₂ sensor, the inventors prefer the VAYU Meter, which is a very accurate non-invasive meter that provides very sensitive measurements of CO₂ concentration in the exhaust of a fermentation vessel. U.S. Pat. No. 9,441,260, assigned to the parent company of the applicant of the present application, describes the method used by a processor to determine the CO₂ concentration in the fermentation vessel from the measured CO₂ concentration in the exhaust pipe. The Vayu Meter is manufactured by the applicant of the present application. Embodiments of the VAYU Meter are described in detail in co-pending international patent application number PCT/IL2019/050750 to the applicant of the present application. The VAYU Meter is coupled to a controller that comprises a processor, a data storage device, and a graphic user interface. The VAYU Meter provides a real time output control via analog/digital connection. The VAYU Meter comprises an infrared laser, detector and optical components configured to provide identical optical paths through the gases that exit the fermenter, thereby enabling continuous metabolic gas detection for highly sensitive monitoring of the process in any size fermenter with the same optical path. The VAYU Meter records and analyzes CO₂ metabolic gas concentrations produced during the respiration and growth of living cells. Continuous, automatic measurements via the IR optical system allow in-situ detection of metabolic gases without interrupting the fermentation process for invasive sampling.

FIG. 5 is a graph comparing yield of fed-batch produced product for production runs carried out by following the protocol previously used (lower curve) and by using the disclosed system, method and controller for feeding the carbon source only. The graph shows as increase in yield above 20% and a potential savings of time of approximately 24 hours.

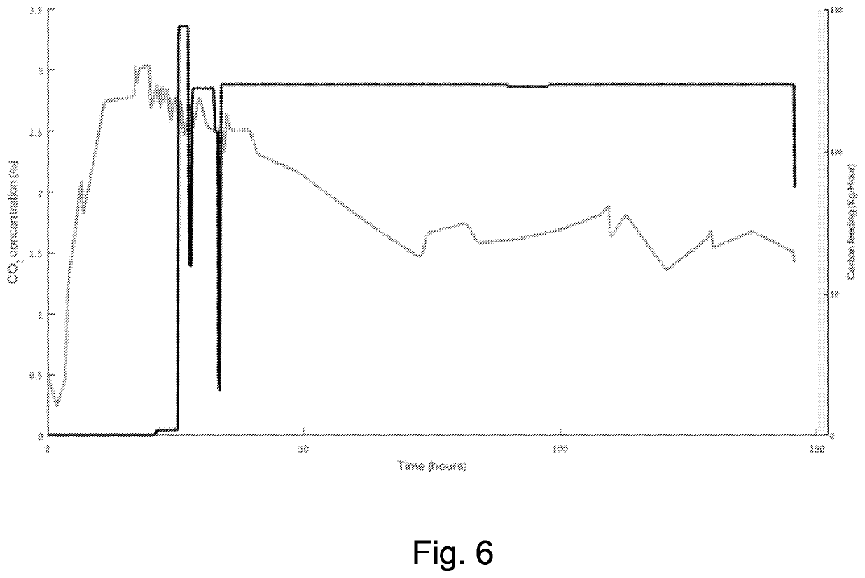

FIG. 6 shows graphs of CO₂ concentration (gray curve) and carbon source feeding (black curve) as functions of amount of carbon source feed vs. time during a production run for a secondary derivative in which the carbon source was fed regardless the amount of biomass in the culture according to the standard protocol followed for production of the product. According to the protocol, during the production stage of the process, starting at about 24 hours until the process is terminated, the carbon source is fed in fixed predetermined constant amounts according to a fixed predetermined schedule at a constant rate.

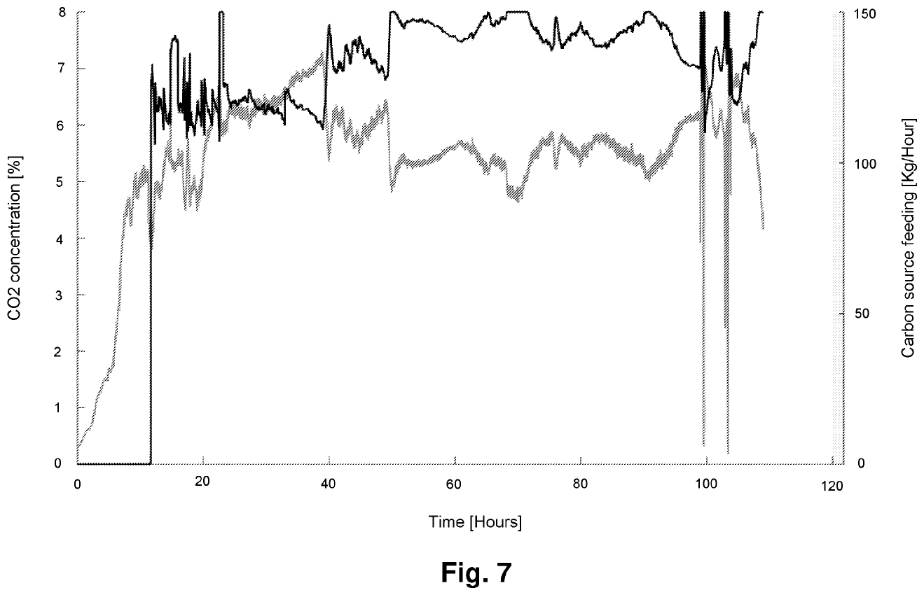

FIG. 7 shows graphs of CO₂ concentration (gray curve) and carbon source feeding (black curve) as functions of time during a production run for the same secondary derivative as in FIG. 6. In FIG. 7 the carbon source was fed according to a PID controller that was using set point and bias values that were calculated for feeding according to CO₂ only according to part of the method described herein above. In the production run shown in this figure, the carbon source was fed with opposite correlation to the culture state according to the CO₂ concentration using a close loop feedback control—when CO₂ went up less carbon source was added and when CO₂ went down more carbon source was added with the amounts of carbon source depending on the deviation of the instantaneous value of the CO₂ concentration from the time varying value of the set point derived from the PID controller.

Comparison of FIG. 6 with FIG. 7 illustrates some of the advantages of the present method over the traditional protocol. In particular during most of the production stage of the process the CO₂ concentration in FIG. 7 is constant indicating equilibrium between cell growth and death and ideal conditions for product formation. In contrast, in FIG. 6 the CO₂ concentration during the production phase is very uneven indicating conditions that are not conducive to optimal production of product. Also referring to FIG. 7 the sever drop in CO₂ level is followed immediately by a large feed of carbon source after which there is an immediately rise in the CO₂ level followed by a rapid drop in CO₂. Another injection of carbon source again briefly raises the CO₂ concentration, which again falls rapidly after the carbon source feeding ceases and continues to drop even when carbon source is added between about 110 and 115 hours. This behavior of the CO₂ concentration indicates that the process should be terminated at about 115 hours. This is in stark contrast to FIG. 6, where the protocol dictates termination of the process at 150 hours.

In one or more embodiments, methods as herein disclosed include a first off-line stage of building a mathematical model. The model is a mathematical description which comprises both controlled and monitored parameters. The main guidelines for generating the model are the academic literature and good fitness of the model to data measured in experimental runs of the process.

Following the building of the model a machine learning based training phase is conducted off line with the goal of creating a trained agent capable of making state dependent decisions (actions), which will eventually optimize the process according to predetermined goals that are determined by the customer, for example: achieving one or more of high yield, low impurities, and time reduction.

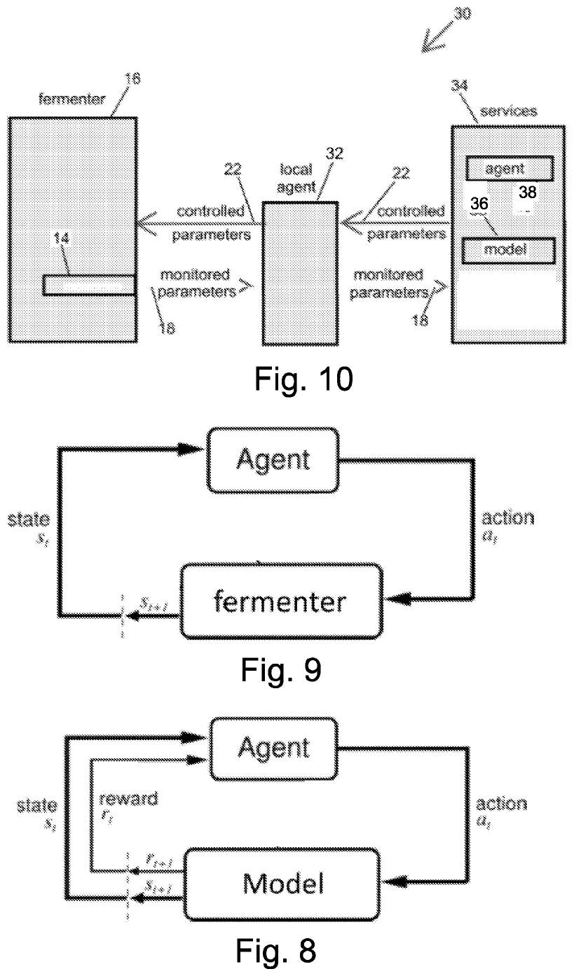

FIG. 8 schematically shows the reinforcement learning iterative training phase, wherein each cycle represents one time step or an episode of several time steps of the training. If, for example, the time step is one minute and the episode is 10,000 minutes long, then the cycle of FIG. 8 is exemplarily carried out 10,000 times during the learning based training phase. Since the learning based training phase is conducted off line, the actual 10,000 minutes long cycle is executable in silico at fraction of this time, which allows the carry out during the learning based training of a reasonable duration, such as several hours or days, a 10,000 minutes long cycle 10,000 times.

In FIG. 8, Sₜ is the state at time t representing in the present case monitored parameters (e.g. DO concentration, carbon source concentration and nitrogen concentration) generated only by using the model; aₜ is the action calculated by the agent at time t representing in the present case the controlled parameters (e.g. carbon source feeding, nitrogen source feeding and agitation); rₜ is the reward at time t, representing the quality of the action at time t−1. In some cases, rₜ may also the quality of several previous actions, e.g. t−1, t−2, etc. For example, if one of the criteria of performance is yield, then high yield rate reflects an advantageous value of aₜ₋₁, therefore the learning algorithm will increase the probability for the action that caused it in the next episode and low yield rate, on the other hand, will cause a decrease in the probability of this action.

In order to achieve high performance, the learning process requires a large data set to learn from. In a specific embodiment of the method, the machine learning technique used during the training phase to generate the agent is reinforcement learning (RL). While most machine learning algorithms use prefabricated data sets, reinforcement learning as herein disclosed uses a mathematical model describing the process to generate an unlimited amount of artificial data. In this case, the RL algorithm does not use monitored parameters measured in fermenter, but the RL algorithm uses the model to generate monitored parameters, represented by Sₜ, to be used in a following episode based on the reward it determines for the process run based on the parameters that it had generated in the previous episode. In each cycle, the controlled parameters are calculated according to the current agent, represented by aₜ. For the first few episodes, arbitrary values of the parameters are entered into the algorithm in order to initiate the iterative learning process. During the training phase, the training consists of a large number of consecutive episodes. In one specific example, about 20,000 episodes were required; but in general, for different processes, more numerous or fewer episodes might be required to achieve the desired performance.

An episode is a simulated way to predict a whole real fermentation process with controlled parameters of each episode determined using the agent achieved from all previous episodes. All of the episodes are governed by the same model, but each episode differs from the others by its unique protocol, i.e. action, for each time step. The updates of the agent, namely the changes in weight values in the decision policy, i.e. improving the probability of an action leading to a higher reward (or vice versa) leads to an iterative improvement of the reward value, meaning better goal values, e.g. higher yield, lower impurity, shorter fermentation time, etc. During the training stage based on the rₜ feedback, the agent is being iteratively improved. This upgrade stops when the agent reaches sufficient, optionally maximal performance, which occurs when the agent achieves repetitive high reward values for simulated runs. In one or more embodiments, the model does not change during the training stage of the agent; however, the model has been developed for a specific fermentation process. For a different process the algorithm that is responsible for training the agent is unchanged; however, the model will change as well as the action and the system state. These differences will force a completely new training process.

FIG. 9 schematically shows control of the fermenter by the trained agent during live production runs. After the training phase, monitored parameters may no longer be calculated by the model but are measured by sensors located in the fermenter. Nevertheless, the algorithms of the agent may have been trained using a model that comprises parameters for which live measurements aren't available during production runs, e.g. parameters that have to be measured off-line such as carbon or nitrogen source concentration determined by titration. To deal with this situation parameters that don't have live measurements are simulated using the model and are sent to the agent during live batches. This option enables sending as detailed data as possible to the agent at any time. In FIG. 9, Sₜ is the state at time t, representing the monitored parameters measured by sensors in the fermenter (and simulated by the model if necessary). The state is sent to the agent that uses, for example, a deep neural network (DNN), which has been trained to optimize the process by, for example, increasing yield, decreasing impurity and short fermentation duration. aₜ is the action calculated by the agent at time t, i.e. the values of controlled parameters that are sent to the fermenter.

FIG. 10 schematically shows an embodiment of a closed loop system 30 configured for carrying out an embodiment of the method for optimizing controlled parameters in an example fermentation process. The system 30 is comprised of three main units: fermenter 16 containing sensors 14; services (controller) 34, which comprises an agent 38 that comprises an algorithm trained to find the optimal action to take at a particular time based on the system state at that time; and local agent 32, which is a mediator configured to transfer data to and from both the fermenter and services. Optionally, services 34 comprise a digital model 36 that represents the fermentation process being carried out in fermenter 16. As for the first embodiment, monitored parameters 18 are parameters that are measured by sensors 14 in fermenter 16, e.g. CO₂ concentration, nitrogen concentration, dO₂, pH, temperature, air flow, and agitation and controlled parameters are parameters that are allowed to be changed, e.g. feeding of a carbon source, feeding of a nitrogen source, agitation, temperature, and aeration.

Live connection to agent 38 (with or without local agent 32 if a wired communication link between fermenter 16 and services 34 is used) is utilized for troubleshooting, software updating and data withdrawal. It is possible to provide services 34 incorporated in a computer located in the facility housing the fermenter with remote access; however, cloud-based architecture is preferred to provide higher security since the algorithms are not physically located in the costumer's facility, data access, connection speed, and reliability. In the cloud based architecture the local agent 32 encrypts data received from the sensors 14 before sending the data to services 34 and decrypts encrypted data received from services 34 before sending it to fermenter 16.

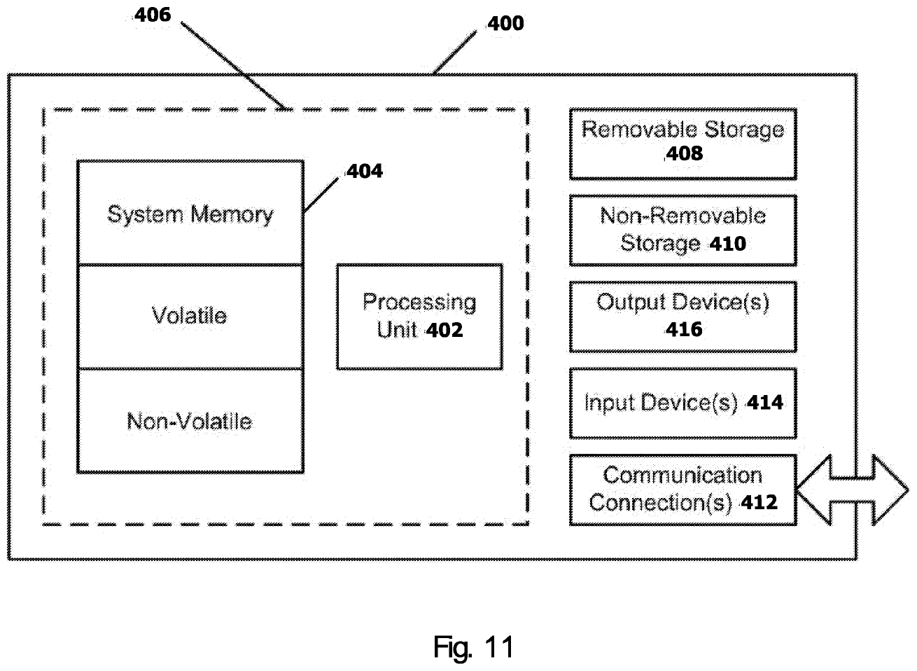

With reference to FIG. 11, an example system for implementing aspects described herein includes a computing device, such as computing device 400. In its most basic configuration, computing device 400 typically includes at least one processing unit 402 and memory 404. Depending on the exact configuration and type of computing device, memory 404 may be volatile (such as random-access memory (RAM)), non-volatile (such as read-only memory (ROM), flash memory, etc.), or some combination of the two. This most basic configuration is illustrated in FIG. 11 by dashed line 406.

Computing device 400 may have additional features/functionality. For example, computing device 400 may include additional storage (removable and/or non-removable) including, but not limited to, magnetic or optical disks or tape. Such additional storage is illustrated in FIG. 11 by removable storage 408 and non-removable storage 410.

Computing device 400 typically includes a variety of computer readable media. Computer readable media can be any available media that can be accessed by computing device 400 and include both volatile and non-volatile media, and removable and non-removable media. Computer storage media include volatile and non-volatile, and removable and non-removable media implemented in any method or technology for storage of information such as computer readable instructions, data structures, program modules or other data.

Memory 404, removable storage 408, and non-removable storage 410 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 400. Any such computer storage media may be part of computing device 400.

Computing device 400 may contain communications connection(s) 412 that allow the device to communicate with other devices. Computing device 400 may also have input device(s) 414 such as a keyboard, mouse, pen, voice input device, touch input device, etc. Output device(s) 416 such as a display, speakers, printer, etc. may also be included. All these devices are well known in the art and need not be discussed at length here.

It should be understood that the various techniques described herein may be implemented in connection with hardware or software or, where appropriate, with a combination of both. Thus, the processes and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

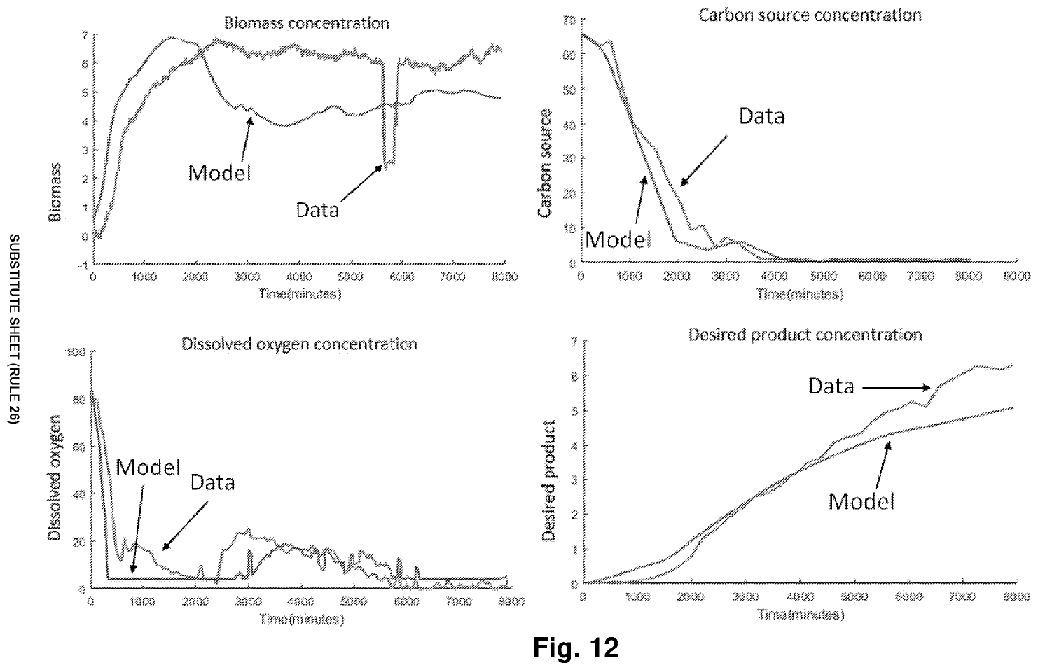

FIG. 12 shows graphs obtained during the model validation step. The graphs illustrate a comparison between the predicted monitored parameters (herein “model”) and real measurements of monitored parameters (herein “data”) of: biomass oxygen, dissolved oxygen, carbon source, and desired product (e.g., an antibiotic). In one or more embodiments, the step of validating the model is conducted by: i) selecting initial input values of monitored parameters and controlled parameters, ii) processing the initial input values by the model to thereby obtain calculated predictive values for the monitored parameters, iii) determining a difference between the calculated predictive values of the monitored parameters and respective values of the monitored parameters as obtained in previous data of the reactor, and iv) further determining that the difference does not exceed a predefined threshold.

In one or more embodiments, the predefined threshold includes one or more values (absolute and/or relative values, e.g., a percentage) for allowing to determine the compatibility of the model for a process of a reactor. The model chosen should mimic the actual dynamic behavior of the reactor such that difference between the calculated predictive values and the respective values of the monitored parameters in the historical data (previous data of a reactor) that does not exceed the predetermined threshold may indicate compatibility of the model.

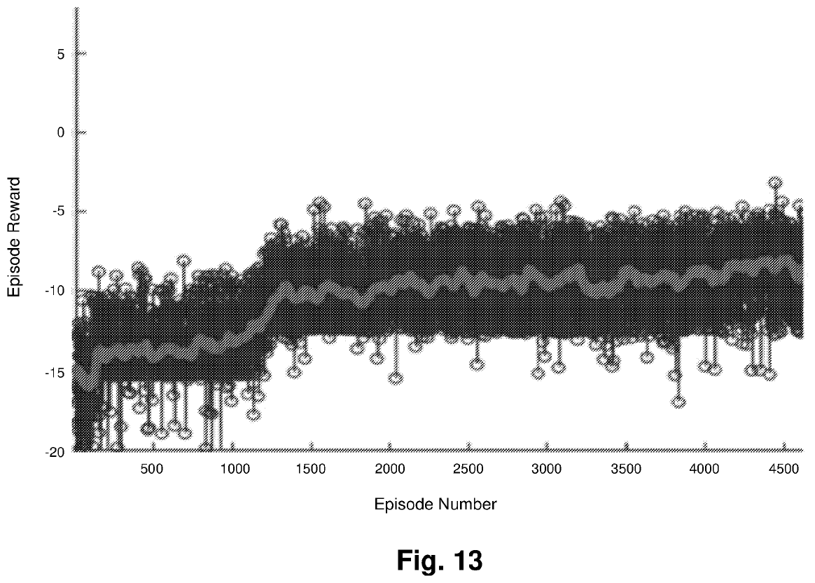

FIG. 13 illustrates the learning process of a reinforcement learning algorithm. X axis denotes the number of episodes executed, Y axis denotes the reward value. The black lines represent specific value of each reward. The central bold line denotes averaging the last 50 episodes, showing the learning trend. This graph displays 4500 episodes of the learning phase where the average reward value continuously improves due to policy update following each episode.

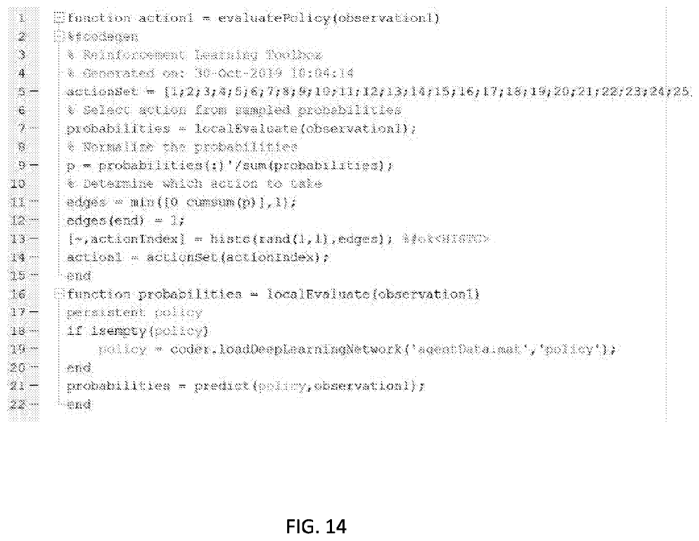

FIG. 14 shows an example computer program code configured for evaluation and/or update of the decision policy or policy function. This function gets as an input the state of the process (“observation1”) at some time point and it returns the action which supposed to be optimal (with high confidence) for that state. The function which appears in lines 16-22 extracts from the file named “agentData.mat” the final policy. This policy helps us to decide on the optimal action for each state (line 21).

Although example implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include PCs, network servers, and handheld devices, for example.

The following are working empirical examples. This following example was performed for the optimization of an antibiotic production process, based on the embodiments described hereinabove. This activity was conducted, with the objective of increasing the yield for the selected fermentation process.

The instant fermentation process concerns a species of Streptomyces bacteria which produces an antibiotic compound.

The fermentation process begun with a small number of bacteria inserted to the fermenter that contained a grow medium. The process was divided into two main phases: (1) growth phase, where the bacteria replicated itself, thus increasing the biomass inside the fermenter; and (2) production phase where the vast majority of production was conducted, and the biomass has not changed dramatically. Each of these phases was composed of several sub-phases which shows different behaviors.

The physical conditions of dissolved oxygen concentration, carbon source concentration, nitrogen source concentration, and pH, were measured using sensors in the fermenter and were selected as monitored parameters. Controlled parameters of carbon source feeding, nitrogen source feeding, agitation and airflow were selected.

The development protocol of the intelligent controller was composed of a combination of constant values to some of the monitored parameters. This protocol was developed using an understanding of the biological properties of the of the process, as well as try and error R&D experiments.

The main objective was to increase the desired antibiotic production, with secondary objectives of decreasing the impurity (relative amount other compounds produced, which making the purification process less efficient). A significant improvement has been achieved by creating an intelligent controller based, as described hereinabove. The controller was activated every predetermined period of time, where the input was a set of monitored parameters and the output was a set of controlled parameters.

A model which describes the dynamics of a single fermentation process was formed. This model contained the dependency of the controlled/monitored parameters given a simulative prediction of the yield obtained in various simulated experiments, differentiated in initial conditions and controlled parameters values.

The mathematical model that was formed included a set of differential equations which collectively comprised parameters describing different aspects of the subject fermentation process. The equations were based inter alia on academic literature, past data collected on the process, and the results of specifically designed experiments. The model contained several parameters which were calibrated based on collected data.

Following the building of the model, a machine learning-based training phase was conducted offline. The training stage included a large amount of simulative processes (episodes). It started with an arbitrarily agent and based on the simulative yield it improved, iteratively, the agent's performances. All of the experiments were governed by the same model, but each episode differed from the others by its unique protocol, i.e. action, for each time step (state). The updates of the agent, i.e. improving the probability of an action leading to a higher reward (or vice versa) led to an iterative improvement of the reward value, with better goal values, in instant case, higher yield and lower impurities. This training stage ended up with a trained (optimal) agent capable of making state dependent decisions (actions).

The model was realistic only for well-defined range of monitored parameters, i.e. the model succeeded to predict the dynamic of monitored parameters as long as these values were within the realistic range. Additional restrictions regarding the controlled and monitored parameters were raised from FDA restrictions and customer request, such as maximum amount of dextrose feeding. As a consequence, restrictions which cancel actions that may lead to such undesired scenarios were introduced.

This agent was embedded in the process (during several experiments) as a controller of the controlled parameters. It showed sufficient results, i.e., it improved the final yield of the process.

Results—the performance of the trained agent was examined during 3 experiments. Each of the experiments was composed from 2 fermentation processes which were executed simultaneously. While one of the fermentation processes was governed by the standard protocol the other was governed by the controller. The average improvement in terms of production yield was around 13%. The minimal improvement was 9%. Thus, at least some embodiments provide an improvement in one or more objectives of a production process by at least about 5%, at least about 7%, or at least 9%.

In this model, t represented the time and the model was updated with a time differential of dt Presented herein are the differential equations of the model.

The biomass trend was given by equation (1):

X(t+1)=X(t)+dt(X(t)(μ(t)−K_{d})) (1)

wherein: X(t) is the biomass concentration in the fermenter at time t, K_{d} is the death factor constant of the cells, and μ(t) is the growth rate of the cells at time t, X(t+1) is the value of X(t) one minute after t, and μ(t) is given by equation (2):

$$μ ( t ) = {μ}_{x} ( \frac{S ( t )}{{K}_{x} + S ( t )} ) ( \frac{C L ( t )}{C L ( t ) + {K}_{o x}} ) ( \frac{A ( t )}{A ( t ) + {K}_{x a}} ) \qquad ( 2 )$$

wherein: μₓ is the maximal growth rate constant of the cells, Kₓ is the carbon source limitation constant for growth, Kₒₓ is the oxygen limitation constant for growth, S(t) is the carbon source concentration in the fermenter at time t, CL(t) is the dissolved oxygen concentration at time t, A(t) is the nitrogen source concentration in the fermenter at time t, and Kₓₐ is the nitrogen source limitation constant for growth.

The production trend was given by equation (3):

P(t+1)=P(t)+dt(μₚₚ(t)X(t)−KP(t)) (3)

wherein: P(t) is the product concentration in the fermenter, K is the product hydrolysis rate constant, and μₚₚ(t) is the production rate at time t, μₚₚ(t) is given by equation (4):

$${μ}_{p p} ( t ) = {μ}_{p} ( \frac{A ( t )}{{K}_{p} + A ( t )} ) ( \frac{C L ( t )}{C L ( t ) + {K}_{o p}} ) ( \frac{S ( t )}{{K}_{I} + S ( t ) + \frac{{s ( t )}^{2}}{{K}_{p s 2}}} ) \qquad ( 4 )$$

wherein, μₚ is the maximal production rate constant, Kₚ is the production inhibition constant for nitrogen source, Kₒₚ is the production inhibition constant for dissolved oxygen, K_{I} is the first inhibition constant for carbon source and Kₚₛ₂ is the second inhibition constant for carbon source.

The carbon source was used for cell growth, production, and maintenance of the fermentation process. The amount of the carbon source in the vessel decreased with time and was increased by feeding during the process. The carbon source trend was given by equation (5):

$$S ( t + 1 ) = S ( t ) + d t [ X ( t ) ( - \frac{μ ( t )}{{Y}_{x / s}} - {m}_{x} - \frac{{μ}_{p p} ( t )}{{Y}_{p / s}} ) + \frac{{S}_{i n} ( t )}{Vessel Volume} ] \qquad ( 5 )$$

wherein, S(t) is the carbon source concentration in the fermenter at time t, Y_{x/s} is the growth yield constant for the carbon source, Y_{p/s} is the production yield constant for the carbon source, mₓ is the maintenance constant of the carbon source, and Sᵢₙ is the carbon source feeding value.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

1-31. (canceled)

32. A method for optimized industrial production using machine learning, comprising:
creating a model defining dependencies among a plurality of parameters for an industrial production process, the plurality of parameters including a plurality of controlled parameters and a plurality of monitored parameters;
training an agent via reinforcement learning based on iterative application of the model, wherein the agent is trained to determine new values for the plurality of controlled parameters based on current values of the plurality of monitored parameters in order to optimize the industrial production process with respect to at least one predetermined objective; and
iteratively modifying, by the trained agent, current values of the plurality of controlled parameters in real-time during operation of the industrial production process.

33. The method of claim 32, wherein training the agent further comprises:
simulating a portion of the plurality of monitored parameters using the model in order to generate artificial data, wherein the agent is trained at least partially using the artificial data.

34. The method of claim 33, wherein the artificial data includes a plurality of artificial parameters, further comprising:
validating the model by comparing the plurality of artificial parameters to a plurality of test parameters measured during a production run of the industrial production process.

35. The method of claim 34, wherein validating the model further comprises:
determining a difference between the plurality of artificial parameters and the plurality of test parameters, wherein the model is validated when the difference is below a threshold.

36. The method of claim 34, wherein validating the model further comprises:
selecting a plurality of input values; and
processing the plurality of input values using the model in order to determine the plurality of artificial parameters, wherein the plurality of test parameters includes historical monitored parameters for the industrial production process.

37. The method of claim 32, wherein training the agent further comprises:
iteratively determining at least one reward, wherein each reward is a score function defined with respect to one of the at least one predetermined objective; and
updating the agent based on the at least one reward determined at each iteration.

38. The method of claim 37, wherein the agent has at least one weight value defining the dependency between the plurality of controlled parameters and the plurality of monitored parameters, wherein updating the agent further comprises:
determining at least one new value for the at least one weight value; and
changing at least a portion of the at least one weight value based on the determined at least one new value.

39. The method of claim 32, further comprising:
dividing the industrial production process into a plurality of phases; and
determining an initial set of controlled parameters for each of the plurality of phases, wherein the plurality of controlled parameters is initialized to the respective initial set of controlled parameters at the beginning of each phase.

40. The method of claim 32, wherein the at least one predetermined objective includes at least one of: high product yield, short fermentation duration, low impurity value, product quality, and process efficiency.

41. A non-transitory computer readable medium having stored thereon instructions for causing a processing circuitry to execute a process, the process comprising:
creating a model defining dependencies among a plurality of parameters for an industrial production process, the plurality of parameters including a plurality of controlled parameters and a plurality of monitored parameters;
training an agent via reinforcement learning based on iterative application of the model, wherein the agent is trained to determine new values for the plurality of controlled parameters based on current values of the plurality of monitored parameters in order to optimize the industrial production process with respect to at least one predetermined objective; and
iteratively modifying, by the trained agent, current values of the plurality of controlled parameters in real-time during operation of the industrial production process.

42. A system for optimized industrial production using machine learning, comprising:
a processing circuitry; and
a memory, the memory containing instructions that, when executed by the processing circuitry, configure the system to:
create a model defining dependencies among a plurality of parameters for an industrial production process, the plurality of parameters including a plurality of controlled parameters and a plurality of monitored parameters;
train an agent via reinforcement learning based on iterative application of the model, wherein the agent is trained to determine new values for the plurality of controlled parameters based on current values of the plurality of monitored parameters in order to optimize the industrial production process with respect to at least one predetermined objective; and
iteratively modify, by the trained agent, current values of the plurality of controlled parameters in real-time during operation of the industrial production process.

43. The system of claim 42, wherein the system is further configured to:
simulate a portion of the plurality of monitored parameters using the model in order to generate artificial data, wherein the agent is trained at least partially using the artificial data.

44. The system of claim 43, wherein the artificial data includes a plurality of artificial parameters, wherein the system is further configured to:
validate the model by comparing the plurality of artificial parameters to a plurality of test parameters measured during a production run of the industrial production process.

45. The system of claim 44, wherein the system is further configured to:
determine a difference between the plurality of artificial parameters and the plurality of test parameters, wherein the model is validated when the difference is below a threshold.

46. The system of claim 44, wherein the system is further configured to:
select a plurality of input values; and
process the plurality of input values using the model in order to determine the plurality of artificial parameters, wherein the plurality of test parameters includes historical monitored parameters for the industrial production process.

47. The system of claim 42, wherein the system is further configured to:
iteratively determine at least one reward, wherein each reward is a score function defined with respect to one of the at least one predetermined objective; and
update the agent based on the at least one reward determined at each iteration.

48. The system of claim 47, wherein the agent has at least one weight value defining the dependency between the plurality of controlled parameters and the plurality of monitored parameters, wherein the system is further configured to:
determine at least one new value for the at least one weight value; and
change at least a portion of the at least one weight value based on the determined at least one new value.

49. The system of claim 42, wherein the system is further configured to:
divide the industrial production process into a plurality of phases; and
determine an initial set of controlled parameters for each of the plurality of phases, wherein the plurality of controlled parameters is initialized to the respective initial set of controlled parameters at the beginning of each phase.

50. The system of claim 42, wherein the at least one predetermined objective includes at least one of: high product yield, short fermentation duration, low impurity value, product quality, and process efficiency.
